(19) United States
(12) Patent Application Publication
(10) Pub. No.: US 2014/0123731 A1
(43) Pub. Date: May 8, 2014

(54) Sensing Condition of Fluids

(71) Applicant: CATERPILLAR, INC., Peoria, IL (US)

(72) Inventor: Amiyo Basu, Peoria, IL (US)

(73) Assignee: CATERPILLAR, INC., Peoria, IL (US)

(21) Appl. No.: 13/672,249

(22) Filed: Nov. 8, 2012

(51) Int. Cl. G01N 33/00

(52) U.S. Cl. CPC G01N 33/00
USPC 73/38

(57) ABSTRACT

A method and system for evaluating a fluid in a machine entails periodically evaluating a permittivity of the fluid and based on periodically evaluating the permittivity of the fluid, observing a change in the permittivity of the fluid. Based on the observed change in the permittivity of the fluid, it is determined whether the fluid is exhausted or contaminated.

### TECHNICAL FIELD OF THE DISCLOSURE

The present disclosure relates to machine fluid quality analysis and, more particularly, relates to a system and method for using the permittivity of a fluid to evaluate it in situ for degradation.

### BACKGROUND OF THE DISCLOSURE

In industry, the use of fluids in machines is widespread; hydraulic systems utilize hydraulic fluid, while engines generally require motor oil to function and many transmissions require transmission fluid to function. However, the conditions under which industrial machines are used are often hostile, both in terms of introducing contaminants to machine fluids as well as in terms of causing degradation of fluids through heat cycling and so on. Because of these various degradation processes, machine fluids are typically changed according to a schedule.

However, changing a machine fluid according to a schedule incurs the risk that the fluid in question may have been subjected to unusually harsh conditions, causing premature breakdown. Similarly, if the fluid in question has been subjected to less strenuous conditions, it may still be functional at the time that the schedule would require replacement, causing waste.

One attempt to address this problem analyzes the capacitance of a fluid to try to determine degradation of the fluid. In particular, U.S. Pat. No. 5,631,568 discloses a system for determining remaining life of hydraulic fluid using a capacitor formed by a pair of electrodes. A charging circuit produces a charging current of constant magnitude. The charging current is used to charge the capacitor to a predetermined voltage. A timing circuit measures the elapsed time between the time at which the charging circuit begins to produce the charging current and the time at which the capacitor has been charged to the predetermined voltage. The timing circuit also produces a pulse width modulated signal. The magnitude of the pulse width modulated signal is indicative of the time difference. A controller receives the pulse width modulated signal and determines the life of the hydraulic fluid.

However, in practice, capacitance measurements prove difficult to take accurately due to timing requirements and variations in the system. Moreover, the system of the '568 patent requires a time-varying probe signal to measure capacitance.

The present disclosure is directed to systems and methods that address one or more of the problems set forth above. However, it should be appreciated that the solution of any particular problem is not a limitation on the scope of this disclosure or of the attached claims except to the extent expressly noted. Additionally, the inclusion of any problem or solution in this Background section is not an indication that the problem or solution represents known prior art except as otherwise expressly noted.

### SUMMARY OF THE DISCLOSURE

In accordance with one aspect of the present disclosure, a method is provided for evaluating a machine fluid including measuring a first permittivity of the fluid at a first time when the machine fluid has a temperature substantially the same as a reference temperature and measuring a second permittivity of the fluid at a second time when the machine fluid has a temperature substantially the same as the reference temperature. A change of the second permittivity from the first permittivity is determined, and based on the change of the second permittivity from the first permittivity a quality of the machine fluid is determined.

In accordance with another aspect of the present disclosure, a system is provided for evaluating a fluid in a machine. The system includes a fluid source, a plate pair associated with the fluid source and located such that fluid exists between the plate pair, a temperature sensor situated adjacent the plate pair, and a charge generator for generating a charge on the plate pair at a reference temperature. The system further includes a field detector for detecting a field across the plate pair at the reference temperature and a processor for determining a permittivity of the fluid based on the generated charge and the detected field and for evaluating whether the fluid is exhausted or contaminated based on the determined permittivity.

In accordance with yet another aspect of the present disclosure, a method for evaluating a fluid in a machine is provided, including periodically evaluating a permittivity of the fluid and based on periodically evaluating the permittivity of the fluid, observing a change in the permittivity of the fluid. Based on the observed change in the permittivity of the fluid, the method determines whether the fluid is exhausted or contaminated.

Other features and advantages of the disclosed systems and principles will become apparent from reading the following detailed disclosure in conjunction with the included drawing figures.

### BRIEF DESCRIPTION OF THE DRAWINGS

FIG. 1 is a partial cut-away simplified fluid circulation system consistent with an aspect of the disclosed principles;

FIG. 2 is a system schematic in accordance with an aspect of the disclosed principles;

FIG. 3 is a sample simulated permittivity plot showing changes in permittivity usable to evaluate the fluid of interest in accordance with an aspect of the disclosed principles; and

FIG. 4 is a flow chart illustrating a process of employing fluid permittivity measurements to evaluate a fluid for contamination or fluid exhaustion.

### DETAILED DESCRIPTION OF THE DISCLOSURE

The present disclosure provides a system and method for analyzing machine fluids in situ to detect excessive degradation of the fluids. In an embodiment, a permittivity sensor is located in a fluid reservoir or conduit. The reservoir may be for example a fluid sump or tank from which fluid is drawn or to which fluid is returned during use. The fluid conduit may be a hose or housing portion used to convey fluid during use.

Permittivity is a property that reflects the resistance encountered when attempting to form an electric field in a medium, with a higher permittivity indicating a larger electrical field in a material for the same applied charge. The permittivity of a fluid is affected by the constituents of the fluid and their individual permittivity.

Thus, for example, a detergent may influence the permittivity in different ways depending upon the point in the detergent's life cycle that the measurement is taken. Similarly, contaminants may progressively affect the permittivity of a fluid. As such, the permittivity of the fluid may be used to measure both degradation and contamination.

Because it affects microscopic mobility and other characteristics related to electric field formation, the temperature at which a permittivity measurement is taken can influence the permittivity measured. For example, a fluid at a higher temperature may exhibit a higher permittivity than that same fluid when measured at a lower temperature.

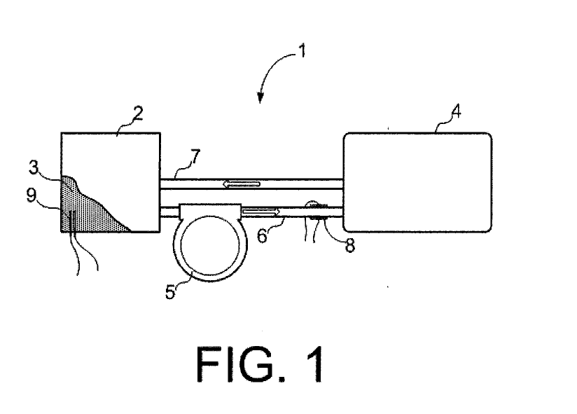

Turning to the figures, FIG. 1 is a simplified partial cut away view of a fluid circulation system in accordance with an aspect of the disclosed principles. The fluid circulation system 1 includes a fluid source and sink, e.g., fluid tank 2. The fluid 3 held in the fluid tank 2 may be any fluid used in a machine such as transmission oil, hydraulic fluid, motor oil or other lubricating fluid, and so on. The fluid circulation system 1 further includes a fluid-using component 4, e.g., a hydraulic system, engine, transmission, and so on.

Within the fluid circulation system 1, the fluid 3 is pumped from the fluid tank 2 via a fluid pump 5. The fluid 3 pumped by the fluid pump 5 is conveyed to the fluid-using component 4 via a first fluid conduit 6. Similarly, the fluid 3 is returned to the fluid tank 2 via a second fluid conduit 7. Though not shown, the fluid 3 may interact with any number of other components during transfer, i.e., boost pumps, coolers, heaters, and so on.

In order to determine the permittivity of the fluid 3, one or more plate pairs are placed in or near the fluid. By way of example, in the illustrated configuration, a first plate pair 8 is located across the first fluid conduit 6, and a second plate pair 9 is located in the fluid tank 2. It will be appreciated that each of these sensors may detect a different permittivity value, however, in an embodiment it is relative and not absolute permittivity values that are used to detect degradation and contamination. In practice, depending upon the system configuration and user preferences, only a single plate pair may be used, and the examples below will consider a single plate pair. It will be appreciated that the same principles apply regardless of the number of plate pairs utilized.

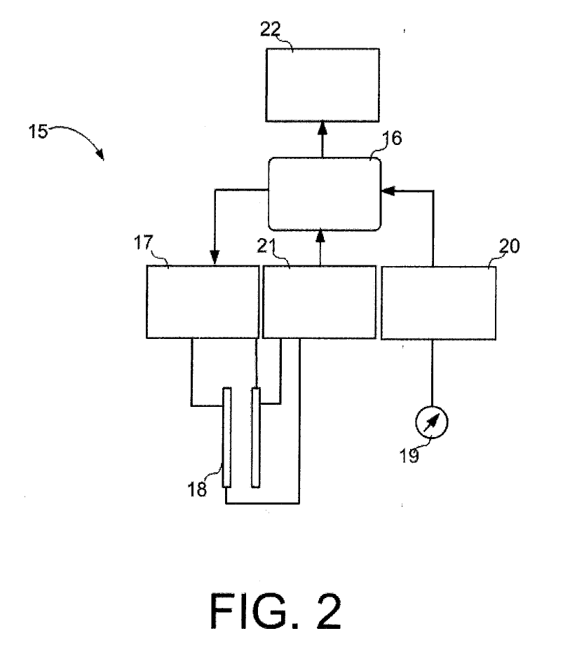

Turning to FIG. 2, this figure shows a simplified schematic for a detection system suitable for performing permittivity readings in a fluid circulation system 1 such as that shown in FIG. 1. The illustrated detection system 15 includes a processor 16 for controlling the actions of the detection system 15. The processor 16 is any suitable processor including a stand-alone processor adapted to serve only the detection system 15 or a multi-use processor such as in an engine controller, transmission controller or other system having a processor for performing various functions.

It will be appreciated that the processor 16 operates by reading computer-executable codes from a computer-readable medium such as a digital memory including, by way of example, a flash drive, disc drive, optical drive, EPROM or other digital memory medium. The processor 16 executes the computer-executable instructions by performing calculations, accepting input, and providing output. Though not necessary, inputs to, and outputs from, the processor 16 are typically digital electronic signals.

The illustrated detection system 15 further includes a charge generator 17 under the control of the processor 16. The role of the charge generator 17 is to generate a voltage across a plate pair 18. The plate pair 18 may be similar to plate pair 8 and plate pair 9 of FIG. 1, and is situated such that a fluid of interest passes between or resides between the plates of the plate pair 18. As such, when the charge generator 17 generates an appropriate voltage (e.g., 50 volts), the voltage is applied across the plates of the plate pair 18 and generates a field within the fluid of interest, the value of the field depending upon the permittivity of the fluid of interest at that time.

The illustrated detection system 15 further includes a temperature sensor 19. The temperature sensor 19 may be of any suitable construction depending upon the implementation, but in an embodiment the temperature sensor is a thermocouple device. The temperature sensor 19 is in thermal contact with the fluid of interest either directly, e.g., via submersion, or indirectly, e.g., via attachment to a housing containing the fluid of interest.

Though not required, in an embodiment, the temperature sensor 19 is placed near the plate pair 18, e.g., a short distance away along the same conduit or submerged in the same tank. In this way, the relative temperature of the fluid at the plate pair 18 can be known during field measurements, such that whatever the temperature may be, it is substantially the same for each measurement.

A temperature sensor interface 20 is provided in the illustrated detection system 15, but may or may not be needed depending upon the type of temperature sensor 19 utilized. In the case of a thermocouple sensor for example, a relatively small voltage is detected by the temperature sensor interface 20 and calibrated to reflect the temperature experienced by the thermocouple. In an embodiment, the calibration or other steps may be performed directly by the processor 16 rather than using a temperature sensor interface 20.

In operation, when the charge generator 17 charges the plate pair 18, the generated electric field is detected by a field detector 21. The field detector 21 may be of any suitable configuration depending upon the implementation environment. In an embodiment, the field detector 21 is an electrostatic detector.

In an embodiment, the processor 16 of the detection system 15 is linked to a user interface 22 for providing feedback to a human operator. Thus, for example, if the permittivity measurements indicate a need to change a fluid of interest, a message can be transmitted to the human operator via the user interface 22. The user interface may also contain a calibration button or selectable element usable to signal the processor 16 that a fluid change has just occurred.

As noted above, the permittivity of the fluid of interest is detected by application of a voltage across the fluid at a reference temperature, and detection of the resultant electric field in the fluid. Repeated permittivity measurements over time at the reference temperature will show the manner in which the fluid's permittivity is changing in response to use and time. The nature of the change in permittivity is then used to indicate the degree of degradation or contamination of the fluid.

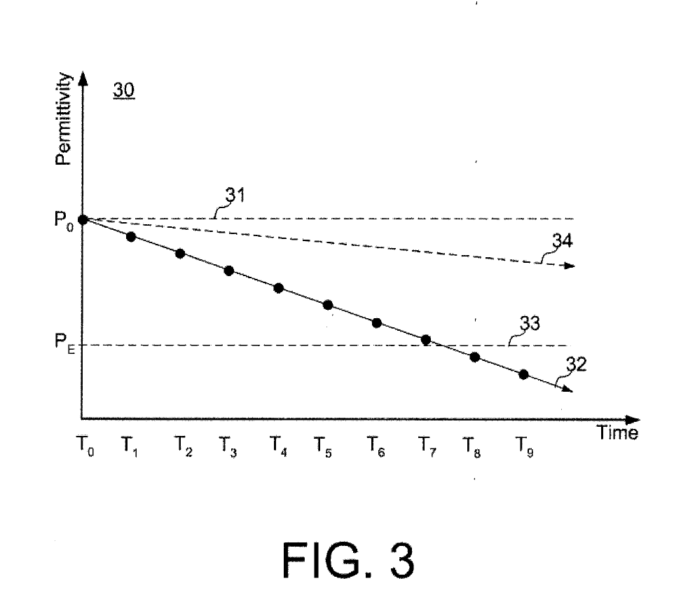

FIG. 3 is an example simulated permittivity plot 30 for motor oil, with permittivity being measured at regular time intervals T₀, T₁, T₂, T₃, T₄, T₅, T₆, T₇, T₈, and T₉. While the time interval for measurement is not critical, in an embodiment, the time interval is a week. In an alternative embodiment the time interval is a day. Each of the measurements of permittivity is taken at a reference temperature of Tᵣ, e.g., Tᵣ=200° F. Thus, for example, at each interval, the measurement of permittivity may be taken when the fluid of interest, in this case motor oil, reaches the reference temperature. In a further or alternative embodiment, the permittivity is measured at a given distance interval in lieu of or in addition to measurement at regular time intervals.

As can be seen from the example simulated permittivity plot 30, the first measurement of permittivity at time T₀ yields a permittivity reading of P₀. This value is reflected in the simulated permittivity plot 30 by first reference line 31. Preferably, the first measurement is taken during the first start up after a fluid change, e.g., by a user or technician selecting a calibration option. The exact value of P₀ is not as important as the later trend in permittivity across the measurement intervals.

As sequential subsequent measurements of permittivity, are taken at points T₁, T₂, T₃, T₄, T₅, T₆, T₇, T₈, and T₉, the permittivity of the motor oil drops approximately linearly, following line 32. It will be appreciated that the change in permittivity is related to changes in motor oil quality, which will depend upon the nature of use and stress to which the motor oil is subjected. As these factors vary, the change in permittivity may be nonlinear though still generally decreasing since degradation of the motor oil does not typically reverse.

As the measurements of permittivity progress over time, and as the permittivity decreases, it will at some point pass the exhaustion limit P_{E} reflected by reference line 33. Interpolating line 32 which generally follows the measurements of permittivity, the permittivity fell below P_{E} after the measurement at T₇ and prior to the measurement at T₈. Thus, at measurement T₈, an alert would be sent to the operator, e.g., via user interface 22, that the motor oil should be changed.

The exhaustion limit P_{E} may be an absolute value of permittivity, however, in an embodiment, the exhaustion limit P_{E} is defined as a percentage of P₀. Thus, for example, P_{E} may be defined as 30% or other appropriate percentage of P₀. In a further embodiment, the value of P_{E} is settable by a user or technician based on experience and/or knowledge of operating conditions.

The line 32 describes the trend of permittivity in the case of degradation of the motor oil. However, contamination of the motor with water, will also impact the permittivity. In particular, contaminants such as water or coolant tend to increase the permittivity of the motor oil. In such a situation, accounting for contemporaneous degradation of the motor oil, the permittivity measurements may show little or no overall decrease in permittivity. Thus, a permittivity change that is much lower than expected may be indicative of contamination.

In the example simulated permittivity plot 30, the limit line 34 is an example of a limit on permittivity change, above which the permittivity measurements may be indicative of contamination. In other words, in the illustrated example, if line 32 fell at or above limit line 34 rather than below it, it may be taken as an indication that the motor oil has become contaminated, e.g., with water or coolant.

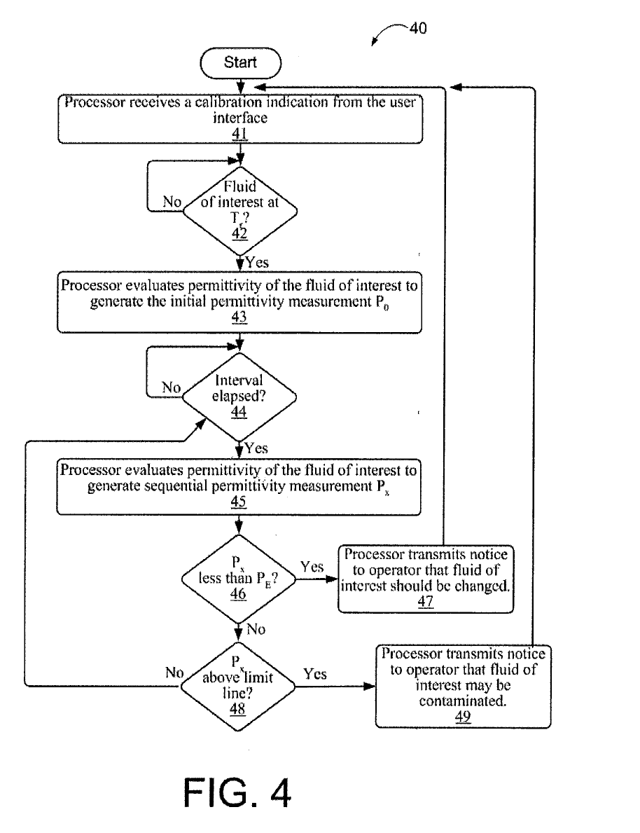

As noted above, the processor 16 facilitates the evaluation of the fluid of interest by executing various steps and actions relative. FIG. 4 is a flow chart showing an example overview process 40 executed by processor 16 to evaluate fluid condition based on permittivity measurements. At stage 41 of the process 40, the processor 16 receives a calibration indication, e.g., from the user interface, that the fluid of interest has been changed. Next at stage 42 of the process 40, the processor 16 checks the temperature sensor and waits for the fluid of interest to reach the predetermined reference temperature Tᵣ. Once the processor 16 determines at stage 42 that the fluid of interest has reached the reference temperature Tᵣ, the processor 16 continues onto stage 43 to evaluate the permittivity of the fluid of interest to generate the initial permittivity measurement P₀.

At stage 44, the processor 16 times the interval to the next measurement. As noted above, the interval may be a predetermined constant interval, e.g., one day, one week, etc. Once it is determined at stage 44 that the time interval to the next measurement has elapsed, the processor 16 takes another permittivity measurement of the fluid of interest at stage 45.

At stage 46, the processor 16 determines whether the permittivity measurement just taken falls below the exhaustion limit P_{E}. If it is determined at stage 46 that the permittivity measurement just taken falls below the exhaustion limit P_{E}, then the process 40 flows to stage 47, whereupon the processor 16 transmits a notification to the user, e.g., via the user interface 22, that the fluid of interest needs to be changed. Otherwise, if it is determined at stage 46 that the permittivity measurement just taken does not yet fall below the exhaustion limit P_{E}, then the process 40 flows to stage 48.

At stage 48, the processor 16 determines whether the permittivity sample just taken falls above the limit line 34. If it is determined at stage 48 that the permittivity sample just taken does fall above the limit line 34, the process 40 flows to stage 49, whereupon the processor 16 transmits a signal to the operator, e.g., via the user interface 22, that the fluid of interest may be contaminated. Otherwise, if it is determined at stage 48 that the permittivity sample just taken does not fall above the limit line 34, the process 40 returns to stage 44 to await expiration of the current measurement interval.

From either of stages 47 and 49, the process 40 may restart and await a calibration signal indicating that the problem transmitted to the operator (fluid exhausted or fluid contaminated) has been resolved and new fluid has been provided.

### INDUSTRIAL APPLICABILITY

In general terms, the present disclosure sets forth a system and method applicable to machines and systems that employ degradable fluids for power transmission, lubrication, cooling, and so on. Fluids used in these and similar applications tend to degrade over time and/or to accumulate contaminants such as water or coolant fluid. As the degradation or contamination progresses, the loss of the original qualities of the fluid of interest can begin to cause damage or performance problems.

In an embodiment, the permittivity of fluids of interest are evaluated periodically to determine changes in the permittivity relative to the initial permittivity of the fluid of interest. Degradation of fluid components such as detergents tends to decrease the permittivity of the fluid, while a heightened concentration of contaminants tends to increase the permittivity of the fluid. Thus, the permittivity of the fluid can be used to determine both degradation and contamination, and to signal the operator of the associated machine when fluids need to be changed or checked for contamination.

The permittivity of different types of fluids differ, i.e., the permittivity of new motor oil may be different than the permittivity of new hydraulic fluid, and so on. In an embodiment, the relative change in permittivity rather than the absolute permittivity is used to evaluate degradation and/or contamination. In a further embodiment, the set point or exhaustion point of various fluids may be set by an operator or technician based on experience or knowledge of working conditions.

It will be appreciated that the present disclosure provides a system and method for evaluating the quality of various fluids used in machine systems such as lubricants and hydraulic fluid. While only certain embodiments have been set forth, alternatives and modifications will be apparent from the above description to those of skill in the art. These and other alternatives are considered equivalents and within the spirit and scope of this disclosure and the appended claims.

What is claimed is:

1. A method for evaluating a machine fluid comprising:
measuring a first permittivity of the fluid at a first time when the machine fluid has a temperature substantially the same as a reference temperature;
measuring a second permittivity of the fluid at a second time when the machine fluid has a temperature substantially the same as the reference temperature; and
determining a change of the second permittivity from the first permittivity and based on the change of the second permittivity from the first permittivity determining a quality of the machine fluid.

2. The method for evaluating a machine fluid according to claim 1, wherein determining a change of the second permittivity from the first permittivity comprises determining that the second permittivity is less than the first permittivity by a predetermined percentage of the first permittivity.

3. The method for evaluating a machine fluid according to claim 2, further comprising notifying an operator that the fluid is exhausted based on determining that the second permittivity is less than the first permittivity by a predetermined percentage of the first permittivity.

4. The method for evaluating a machine fluid according to claim 2, wherein the predetermined percentage is set by an operator.

5. The method for evaluating a machine fluid according to claim 1, wherein determining a change of the second permittivity from the first permittivity comprises determining that the change of the second permittivity from the first permittivity is less than a reference level of change.

6. The method for evaluating a machine fluid according to claim 5, further comprising notifying an operator that the fluid is contaminated based on determining that the change of the second permittivity from the first permittivity is less than a reference level of change.

7. The method for evaluating a machine fluid according to claim 1, wherein each of measuring a first permittivity of the fluid at a first time and measuring a second permittivity of the fluid at a second time includes measuring the permittivity via a plate pair.

8. The method for evaluating a machine fluid according to claim 7, wherein the plate pair is submerged in a tank of the fluid.

9. The method for evaluating a machine fluid according to claim 7, wherein the plate pair is on a conduit for conveying the fluid.

10. The method for evaluating a machine fluid according to claim 1, wherein the fluid is one of a lubricating fluid and a hydraulic fluid.

11. A system for evaluating a fluid in a machine, the system comprising:
a fluid source;
a plate pair associated with the fluid source and located such that fluid exists between the plate pair;
a temperature sensor situated adjacent the plate pair;
a charge generator for generating a charge on the plate pair'at a reference temperature;
a field detector for detecting a field across the plate pair at the reference temperature; and
a processor for determining a permittivity of the fluid based on the generated charge and the detected field and for evaluating whether the fluid is exhausted or contaminated based on the determined permittivity.

12. The system for evaluating a fluid in a machine according to claim 11, wherein the processor is configured to evaluate whether the fluid is exhausted or contaminated based on the determined permittivity by determining a change of permittivity.

13. The system for evaluating a fluid in a machine according to claim 12, wherein the processor is configured to determine a change of permittivity by determining whether the permittivity has decreased by a predetermined percentage.

14. The system for evaluating a fluid in a machine according to claim 13, wherein the processor is configured to notify an operator that the fluid is exhausted when the permittivity has decreased by a predetermined percentage.

15. The system for evaluating a fluid in a machine according to claim 14, wherein the predetermined percentage is set by an operator.

16. The system for evaluating a fluid in a machine according to claim 13, wherein the processor is configured to determine that the fluid is contaminated when the change of permittivity is less than a reference level of change.

17. The system for evaluating a fluid in a machine according to claim 13, wherein the processor is configured to notify an operator that the fluid is contaminated when the change of permittivity is less than a reference level of change.

18. The system for evaluating a fluid in a machine according to claim 11, wherein the plate pair is submerged in a tank of the fluid.

19. The system for evaluating a fluid in a machine according to claim 11, wherein the plate pair is on a conduit for conveying the fluid.

20. A method for evaluating a fluid in a machine comprising:
periodically evaluating a permittivity of the fluid;
based on periodically evaluating the permittivity of the fluid, observing a change in the permittivity of the fluid;
based on the observed change in the permittivity of the fluid, determining whether the fluid is exhausted or contaminated.
